(19) United States
(12) Patent Application Publication
(10) Pub. No.: US 2019/0390150 A1
(43) Pub. Date: Dec. 26, 2019

(54) BIOLOGICAL REACTION DEVICE IN WHICH MICRO-NANO BUBBLES ARE USED, AND BIOLOGICAL REACTION METHOD IN WHICH SAID BIOLOGICAL REACTION DEVICE IS USED

(71) Applicant: Mitsubishi Chemical Engineering Corporation, Chuo-ku (JP)

(72) Inventors: Masamori HIGUCHI, Chuo-ku (JP); Nobuhide KUNITOMO, Chuo-ku (JP); Yuichi KOBAYASHI, Chuo-ku (JP); Kazunori KUMADA, Chuo-ku (JP); Nobuhiro TANAKA, Chuo-ku (JP)

(73) Assignee: Mitsubishi Chemical Engineering Corporation, Chuo-ku (JP)

(21) Appl. No.: 16/471,297

(22) PCT Filed: Jan. 19, 2018
(86) PCT No.: PCT/JP2018/001489

§ 371 Date: Jun. 19, 2019

(30) Foreign Application Priority Data
Jan. 25, 2017 (JP) 2017-011587

(51) Int. Cl. C12M 1/04; C12M 1/00; C12M 1/34; B01D 69/08; B01D 65/08; B01D 53/22

(52) U.S. Cl. CPC C12M 27/04; C12M 29/18; C12M 41/40; C12M 29/04; B01D 2053/224; B01D 69/08; B01D 65/08; B01D 53/22; C12M 29/24

(57) ABSTRACT

Provided are: a biological reaction device that reduces stress and damage visited on microorganisms, etc., in a biological reaction, and enables a biological reaction in which microorganisms, etc., are used to be efficiently and economically performed; and a biological reaction method in which the biological reaction device is used. In order to reduce the stress and damage visited on microorganisms, etc., the amount per minute of a biological culture solution that is extracted from a culture tank and then returned to the culture tank after being admixed with micro-nano-bubbles is set as at least 1% to less than 48% of the biological culture solution accommodated in the culture tank, and the reduction in the dissolved oxygen concentration associated therewith is compensated for by setting the oxygen partial pressure of a gas phase in the upper part of the biological culture solution in the culture tank as at least 0.23 atm to less than 0.6 atm and/or setting the pressure of the gas phase in the upper part of the biological culture solution in the culture tank to at least 1.1 atm to less than 3.0 atm.

### TECHNICAL FIELD

The invention relates to a biological reaction device for culturing aerobic or facultative anaerobic microorganisms (hereinafter, also referred to as “microorganisms, etc.”) to cause the microorganisms, etc., to produce a reaction product in the microorganisms, etc., and to proliferate and a biological reaction method in which the biological reaction device is used. A biological culture solution containing microorganisms, etc., is admixed with micro-nano-bubbles formed from a gas having an increased oxygen content ratio (hereinafter, the “micro-nano-bubbles” may be referred to as “MNB”; “nano-bubbles” are referred to as “NB”; and the “micro-nano-bubbles formed from a gas having an increased oxygen content ratio” may be referred to as “oxygen-enriched MNB”), whereby a biological reaction is efficiently performed.

### BACKGROUND ART

Since biological reactions are slow reactions themselves but do not use enormous energy and many chemical substances unlike chemical reactions, the biological reactions are mild and meaningful reactions for the environment.

However, the biological reactions generally have a problem that the reactions are slow. That is, while the chemical reactions within 1 hour are often sufficient, the biological reactions for several hours to several days if long, or several weeks or more if particularly long may be required. For this reason, the biological reactions are required to be efficiently and economically performed.

As techniques for improving the efficiency of the biological reactions, Patent Documents 1 to 3 disclose that the activation of microorganisms etc., is promoted by causing MNB or NB formed from air to be present in a culture solution in the culture of the microorganisms etc., to achieve the reaction efficiency of the biological reactions and the shortening of a reaction time, etc.

Specifically, Patent Document 1 describes that a culture solution is mixed with MNB and NB of air at a stage before supplying the culture solution to a culture tank. Patent Document 2 describes that a culture solution is mixed with MNB of air at a stage before supplying the culture solution to a culture tank. Patent Document 3 describes that a culture solution is extracted from a culture tank in a batch system, and filtered with a bacteria cell filter to obtain a filtrate, and the filtrate is mixed with MNB of air, and returned to the culture tank.

However, in the devices in which the culture solution supplied to the culture tank is admixed with MNB and NB of air as disclosed in Patent Documents 1 and 2 described above, the culture solution in the culture tank can be admixed with an appropriate amount of MNB and/or NB at the initial stage of the biological reaction, but the content of MNB and/or NB in the culture solution in the culture tank cannot be properly maintained in the whole long-term biological reaction. Therefore, the reaction efficiency of the biological reaction and the shortening of the reaction time, etc., cannot be sufficiently achieved.

As shown in FIG. 9 in Patent Document 3, a device is described, in which the culture solution is extracted from a culture tank 107 as a biological reaction tank, and filtered by a bacteria cell filter 110 to obtain the filtrate, and the filtrate is mixed with MNB of air generated by an MNB generator 116 in a MNB generation tank 115, to return the filtrate to the culture tank. The device can maintain the MNB content of the culture solution in the culture tank at an appropriate value. However, stress and damage are visited on microorganisms etc., in the process of extracting the culture solution from the culture tank, the process of filtering the culture solution with a bacteria cell filter, and the process of returning the culture solution excluding the filtrate to the culture tank, etc., which causes a problem that the activity of the microorganisms etc., is deteriorated.

Then, the inventors found that the oxygen content ratio of a gas forming MNB is set to be higher than an oxygen content ratio in air (about 21%), whereby the following merits are provided, and filed a PCT application earlier: a ratio (hereinafter, also referred to as a “return ratio”) of the amount per minute (hereinafter, also referred to as a “return amount”) of the biological culture solution extracted from the culture tank, admixed with micro-nano-bubbles, and then returned to the culture tank, to the amount of the biological culture solution accommodated in the culture tank can be suppressed to a low level; high concentration oxygen that is easy to be absorbed in the state of MNB can be supplied to microorganisms etc., even when the amount of MNB contained in the biological culture solution in the culture tank is reduced; and the activity of the microorganisms etc., can be maintained. (PCT Application Number: PCT/JP2016/059728, National Phase Application Number to Japan: Japanese Patent Application No. 2016-518206)

In order to reduce the stress and damage visited on the microorganisms, etc., the return rate is required to be suppressed to a low level. This causes a reduced amount of MNB contained in the biological culture solution in the culture tank to cause a reduced dissolved oxygen concentration in the biological culture solution (hereafter, also referred to as a “dissolved oxygen concentration”). In order to compensate for the reduction in the dissolved oxygen concentration, the present inventors have found that not only means for setting the oxygen content ratio of the gas forming MNB to be higher than an oxygen content ratio in air (about 21%) as in the previous invention, i.e., “at least 23% to less than 60%” but also means for setting the oxygen partial pressure of a gas phase in an upper part of the biological culture solution in the culture tank (hereafter, also referred to as “a gas phase in the culture tank”) to be higher than the oxygen partial pressure of air (about 0.21 atm) at normal pressure, i.e., “at least 0.23 atm to less than 0.6 atm”, and/or means for setting the pressure of the gas phase in the culture tank to be higher than normal pressure (1 atm), i.e., at least 1.1 atm to less than 3.0 atm can also be employed, and have completed the invention.

The invention has the following great merits:

Even when the return ratio is suppressed to a low level, and the amount of MNB contained in the biological culture solution in the culture tank is reduced, by increasing the oxygen partial pressure of the gas phase in the culture tank and/or by increasing the pressure of the gas phase in the culture tank, the dissolved oxygen concentration can be maintained without being reduced;

By suppressing the return ratio to a low level, the stress and damage visited on the microorganisms, etc., can be reduced, and energy required for circulating the biological culture solution can be reduced; and

By reducing the amount of MNB contained in the biological culture solution, energy required for driving the MNB generator can be reduced;

The return ratio is suppressed to a low level, whereby a positive displacement pump such as a tube pump, a diaphragm pump, a screw pump, or a rotary pump that causes relatively less stress and damage applied to microorganisms, etc., can be preferably used as a pump for circulating the biological culture solution to the outside of the culture tank.

### CITATION LIST

### Patent Document

Patent Document 1: Japanese Patent No. 4805120

Patent Document 2: Japanese Patent No. 4956052

Patent Document 3: Japanese Patent No. 4146476

### SUMMARY OF INVENTION

### Technical Problem

An object of a biological reaction device of the invention and a biological reaction method in which the biological reaction device is used is to reduce stress and damage visited on microorganisms, etc., in a biological reaction, and to enable the biological reaction in which the microorganisms, etc., are used to be efficiently and economically performed.

### Solution to Problem

In order to solve the above problems, a biological reaction device of the invention and a biological reaction method in which the biological reaction device is used are characterized in that, in order to reduce stress and damage visited on microorganisms, etc., a return amount is suppressed to a low level of “at least 1% to less than 48% of the biological culture solution accommodated in the culture tank per minute”, and the reduction in a dissolved oxygen concentration associated therewith is compensated for by using the following means:

1) means for setting the oxygen partial pressure of the gas phase in the culture tank to be higher than the oxygen partial pressure of air (about 0.21 atm) at normal pressure, i.e., “at least 0.23 atm to less than 0.6 atm”; and/or

2) means for setting the pressure of the gas phase in the culture tank to be higher than normal pressure (1 atm), i.e., “at least 1.1 atm to less than 3.0 atm”. Furthermore, preferably, 3) means for setting the oxygen content ratio of a gas forming MNB to be higher than an oxygen content ratio in air (about 21%), i.e., “at least 23% to less than 60%” may be used in combination with the means of the above 1) or 2).

A positive displacement pump such as a tube pump, a diaphragm pump, a screw pump, or a rotary pump that causes relatively less stress and damage applied to the microorganisms, etc., is preferably used as a pump for circulating the biological culture solution to the outside of the culture tank, whereby the problems can be further solved.

In the invention, the “return ratio (%)”, that is, “the ratio of the return amount to the amount of the biological culture solution accommodated in the culture tank (%)” means a ratio of a volume (% by volume), and “the oxygen content ratio (%)” in the invention means a ratio of oxygen contained in the intended gas (mol %).

### Advantageous Effects of Invention

In the invention, the use of the means of the above 1) and/or 2) makes it possible to maintain the dissolved oxygen concentration without reducing the dissolved oxygen concentration even when the return ratio is suppressed to a low level in order to reduce the stress and damage visited on the microorganisms, etc., whereby the activity of the microorganisms, etc., can be enhanced. Furthermore, preferably, 3) means for setting the oxygen content ratio of the gas forming MNB to be higher than the oxygen content ratio in air (about 21%), i.e., “at least 23% to less than 60%” is used in combination with the means of the above 1) or 2), whereby the above effect can be further exhibited.

Furthermore, the return ratio is suppressed to a low level, whereby the stress and damage visited on the microorganisms, etc., can be reduced, and energy required for circulating the biological culture solution can be reduced.

Furthermore, energy required for driving an MNB generator can be reduced by reducing the amount of the MNB contained in the culture solution.

Furthermore, the return ratio is suppressed to a low level, whereby the positive displacement pump such as a tube pump, a diaphragm pump, a screw pump, or a rotary pump that causes relatively less stress and damage applied to the microorganisms, etc., can be preferably used as a pump for circulating the biological culture solution to the outside of the culture tank, which also can further reduce the stress and damage visited on the microorganisms, etc.

Thus, the invention enables the biological reaction in which the microorganisms, etc., are used to be efficiently and economically performed, which is excellent.

### BRIEF DESCRIPTION OF DRAWINGS

FIG. 1 is a schematic view showing a first embodiment of a biological reaction device of the invention.

FIG. 2 is a cross-sectional view schematically showing a water flow type MNB generator used in the first embodiment.

FIG. 3 is a cross-sectional view schematically showing oxygen enriching means used in the first embodiment.

FIG. 4 is a schematic view showing a second embodiment of the biological reaction device of the invention.

FIG. 5 is a schematic view showing a third embodiment of the biological reaction device of the invention.

FIG. 6 is a schematic view showing a fourth embodiment of the biological reaction device of the invention.

FIG. 7 is a schematic view showing a fifth embodiment of the biological reaction device of the invention.

FIG. 8 is a schematic view showing a biological reaction device used in Reference Examples and Reference Comparative Examples of the present specification.

FIG. 9 is FIG. 1 of Patent Document 3 (Japanese Patent No. 4146476) that is a conventional example.

### DESCRIPTION OF EMBODIMENTS

Hereinafter, embodiments of the invention will be described in detail with reference to the accompanying drawings, but the invention is not limited thereto.

First, general items of a biological reaction device and biological reaction method of the invention will be described.

### Biological Reaction Device of the Invention and Biological Reaction in which Biological Reaction Device is Used

The biological reaction device of the invention and the biological reaction in which the biological reaction device is used can be applied to not only production of reaction products due to microorganisms, etc., such as production of foods, medicines, and chemicals, etc., due to brewing and fermentation etc., and production of bioethanol using biomass but also proliferation of the microorganisms, etc.

The biological reaction of the invention causes microorganisms etc., to produce a reaction product or proliferates the microorganism etc., in a culture solution containing the microorganisms, etc., accommodated in a culture tank, using the culture solution as a nutrient source.

As the culture solution in the invention, one containing saccharides and a nitrogen source is used. Saccharides such as maltose, sucrose, glucose, fructose, and mixtures thereof are normally used. The concentration of the saccharides in the culture solution is not particularly limited, and is preferably 0.1 to 10 w/v %. As the nitrogen source, ammonium chloride, ammonium sulfate or corn steep liquor, yeast extract, meat extract, and peptone, etc., are used. The concentration of the nitrogen source is preferably 0.1 to 10 w/v %. Furthermore, it is preferable to add vitamins and inorganic salts, etc., to the culture solution as needed, in addition to the saccharides and the nitrogen source.

In addition to aerobic and facultative anaerobic microorganisms such as koji molds including Aspergillus bacteria, and Bacillus natto, acetic acid bacteria, yeast bacteria, and lactic acid bacteria, conventionally used in technical fields such as brewing and fermentation, a variety of aerobic and facultative anaerobic microorganisms produced by the gene recombination technique can be used as the microorganisms in the invention. Examples of the cells include animal cells for producing physiologically active peptides or proteins used as antibody drugs, especially gene-modified animal cells.

The concentration of the microorganism, etc., added to the culture solution is not particularly limited, and preferably 0.5 to 10 g/L, and more preferably 3.0 to 6.0 g/L.

Next, the features of the biological reaction device and biological reaction method of the invention will be described.

### MNB Used in Biological Reaction Device of the Invention and Biological Reaction in which Biological Reaction Device is Used

“MNB” used in the biological reaction device of the invention and the biological reaction in which the biological reaction device is used means “micro-bubbles” and/or “nano-bubbles”. While “normal bubbles” rapidly rise in water and burst and disappear on the surface, minute bubbles called “micro-bubbles” and having a diameter of 50 μm or less shrink and disappear in water. At this time, together with free radicals, “nano-bubbles” that are extremely small bubbles having a diameter of 100 nm or less are generated. The “nano-bubbles” remain in water for a relatively long time.

In the invention, air bubbles having a number average diameter of 100 μm or less are called “micro-bubbles”, and air bubbles having a number average diameter of 1 μm or less are called “nano-bubbles”. As a method of measuring the bubble diameters of the micro-bubbles, an image analysis method, a laser diffraction scattering method, an electrical detection band method, a resonance type mass measuring method, and an optical fiber probe method, etc., are generally used. As a method of measuring the bubble diameters of the nano-bubbles, a dynamic light scattering method, a Brownian motion tracking method, an electrical detection band method, and a resonance type mass measuring method, etc., are generally used.

The nano-bubbles that are extremely small bubbles are also called “ultra-fine-bubbles”. At present, ISO (International Organization for Standardization) has considered the creation of international standard for fine bubble technology, and if the international standard is created, the term “nano-bubbles” that is generally used at present may be unified to “ultra-fine-bubbles”.

As an MNB generator, known or commercially available devices can be used. For example, there can be used a “pressure-dissolving micro-bubble-generator” dissolving a sufficient amount of gas in water at certain high pressure and then releasing the pressure, to produce the supersaturation condition of the dissolved gas, and a “loop flow type bubble generation nozzle” utilizing a phenomenon that water flow is caused to stir and mix a mixed fluid containing a liquid and a gas as loop-like flow, and bubbles are subdivided due to turbulent flow generated by the water flow.

As the nano-bubble-generator, there can be used those described in, for example, JP 2007-312690 A, JP 2006-289183 A, JP 2005-245817 A, JP 2007-136255 A, and JP 2009-39600 A.

It is preferable to use a water flow type MNB generator because the MNB generator can economically generate a large amount of MNB.

### Features of Biological Reaction Device of the Invention and Biological Reaction in which Biological Reaction Device is Used

As described above, the features of the biological reaction device of the invention and the biological reaction method in which the biological reaction device is used are mainly based on the following A) and B):

A) in order to reduce the stress and damage visited on the microorganisms, etc., the return rate is suppressed to a low level, and the reduction in the dissolved oxygen concentration associated therewith is compensated for by using 1) means for setting the oxygen partial pressure of the gas phase in the culture tank to be higher than the oxygen partial pressure of air (about 0.21 atm) at normal pressure, i.e., “at least 0.23 atm to less than 0.6 atm”, and/or 2) means for setting the pressure of the gas phase in the culture tank to be higher than normal pressure (1 atm), i.e., at least 1.1 atm to less than 3.0 atm; and

B) a positive displacement pump such as a tube pump, a diaphragm pump, a screw pump, or a rotary pump that causes relatively less stress and damage applied to the microorganisms, etc., is preferably used as a pump for circulating the biological culture solution to the outside of the culture tank.

The features of the biological reaction device of the invention and the biological reaction method in which the biological reaction device is used will be described below.

### First Feature Point: Compensation of Dissolved Oxygen Concentration

The first feature point of the invention is that, when the return amount is reduced to “at least 1% to less than 48% of the biological culture solution accommodated in the culture tank per minute” in order to reduce the stress and damage visited on the microorganisms, etc., the amount of MNB contained in the biological culture solution in the culture tank is reduced, and the dissolved oxygen concentration is lowered, but the reduction in the dissolved oxygen concentration is compensated for by using 1) means for setting the oxygen partial pressure of the gas phase in the culture tank to be higher than the oxygen partial pressure of air (about 0.21 atm) at normal pressure, i.e., “at least 0.23 atm to less than 0.6 atm”, and/or 2) means for setting the pressure of the gas phase in the culture tank to be higher than normal pressure (1 atm), i.e., “at least 1.1 atm to less than 3.0 atm”. Furthermore, 3) means for setting the oxygen content ratio of a gas forming MNB to be higher than an oxygen content ratio in air (about 21%), i.e., “at least 23% to less than 60%” may be used in combination with the means of the above 1) or 2).

The upper limit of the oxygen partial pressure of the gas phase in the culture tank of the above 1) is less than 0.6 atm, preferably 0.55 atm or less, more preferably 0.5 atm or less, and most preferably 0.45 atm or less. If the oxygen partial pressure of the gas phase in the culture tank is excessively increased to 0.6 atm or more, the stress and damage visited on the microorganisms, etc., due to the oxidizing action of oxygen increase. The lower limit is 0.23 atm or more, preferably 0.25 atm or more, more preferably 0.27 atm or more, and most preferably 0.30 atm or more. If the oxygen partial pressure of the gas phase in the culture tank is excessively reduced to less than 0.23 atm, the dissolved oxygen concentration is reduced, which makes it difficult to enhance the activity of the microorganisms, etc.

Thus, the oxygen partial pressure of the gas phase in the culture tank is increased to “at least 0.23 atm to less than 0.6 atm”, whereby the dissolved oxygen concentration can be maintained without being reduced even when the return ratio is suppressed to a low level, which makes it possible to enhance the activity of the microorganisms, etc.

The upper limit of the pressure of the gas phase in the culture tank of the above 2) is less than 3.0 atm, preferably 2.75 atm or less, more preferably 2.5 atm or less, and most preferably 2.25 atm or less. If the pressure of the gas phase in the culture tank is excessively increased to 3.0 atm or more, the stress and damage visited on the microorganisms, etc., due to the oxidizing action of oxygen increase. The lower limit is 1.1 atm or more, preferably 1.2 atm or more, more preferably 1.3 atm or more, and most preferably 1.4 atm or more. If the pressure of the gas phase in the culture tank is excessively reduced to less than 1.1 atm, the dissolved oxygen concentration is reduced, which makes it difficult to enhance the activity of the microorganisms, etc.

Thus, the pressure of the gas phase in the culture tank is increased to “at least 1.1 atm to less than 3.0 atm”, whereby the dissolved oxygen concentration can be maintained without being reduced even when the return ratio is suppressed to a low level, which makes it possible to enhance the activity of the microorganisms, etc.

Furthermore, preferably, 3) means for setting the oxygen content ratio of the gas forming MNB to be higher than the oxygen content ratio in air (about 21%), i.e., “at least 23% to less than 60%” is used in combination with the means of the above 1) and/or 2), whereby the above effects can be further exhibited.

Furthermore, the return ratio is suppressed to a low level, whereby the stress and damage visited on the microorganisms, etc., can be reduced, and energy required for circulating the biological culture solution can be reduced.

Furthermore, energy required for driving an MNB generator can be reduced by reducing the amount of the MNB contained in the culture solution.

In the invention,

1) means for setting the oxygen partial pressure of the gas phase in the culture tank to be higher than the oxygen partial pressure of air (about 0.21 atm) at normal pressure, i.e., “at least 0.23 atm to less than 0.6 atm”,

2) means for setting the pressure of the gas phase in the culture tank to be higher than normal pressure (1 atm), i.e., “at least 1.1 atm to less than 3.0 atm”, and

3) means for setting the oxygen content ratio of a gas forming MNB to be higher than an oxygen content ratio in air (about 21%), i.e., “at least 23% to less than 60%”, that may be preferably used in combination with the means of the above 1) or 2) will be described in more detail.

As the means of the above 1), various known means can be used in the invention, and suitable examples thereof include:

1a) a technique of supplying air having pressure higher than normal pressure and having a normal composition (oxygen content ratio: about 21%) to the gas phase in the culture tank, to increase the pressure of the gas phase in the culture tank, thereby increasing the oxygen partial pressure of the gas phase in the culture tank;

1b) a technique of supplying air having normal pressure and having an increased oxygen content ratio to the gas phase in the culture tank, to increase the oxygen content ratio of the gas phase in the culture tank, thereby increasing the oxygen partial pressure of the gas phase in the culture tank; and

1c) a technique of supplying air having pressure higher than normal pressure and an increased oxygen content ratio to the gas phase in the culture tank to increase the pressure of the gas phase and the oxygen content ratio in the culture tank, thereby increasing the oxygen partial pressure of the gas phase in the culture tank.

In the above techniques, the air having normal pressure or pressure higher than the normal pressure, and a normal composition or an increased oxygen content ratio can also be directly supplied to the gas phase in the culture tank, or can also be supplied to the MNB generator as the gas forming MNB.

The pressure of the gas phase in the culture tank can be increased by attaching a known pressure adjusting valve, etc., to an exhaust path from the gas phase in the culture tank. In order to obtain a gas or air having an increased oxygen content ratio, known oxygen enriching means such as a PSA method and a VSA method using an adsorbent, a water electrolysis method, a cryogenic separation method, a membrane separation method, and a chemical adsorption method can be used, and an oxygen-enriched membrane is preferably used from the economical viewpoint.

As the means of the above 2), various known means can be employed, and suitable examples thereof include a technique of supplying a gas having pressure higher than normal pressure to the gas phase in the culture tank as in the techniques of the above 1a) and 1c), to increase the pressure of the gas phase in the culture tank. In this technique, the gas having pressure higher than normal pressure can also be directly supplied to the gas phase in the culture tank, or can also be supplied to the MNB generator as the gas forming MNB.

Next, suitable examples of the means of the above 3) include means for obtaining a gas having an oxygen content ratio increased by using the known oxygen enriching means, and using the gas as the gas forming MNB.

Next, the suppression of the return ratio to a low level will be described.

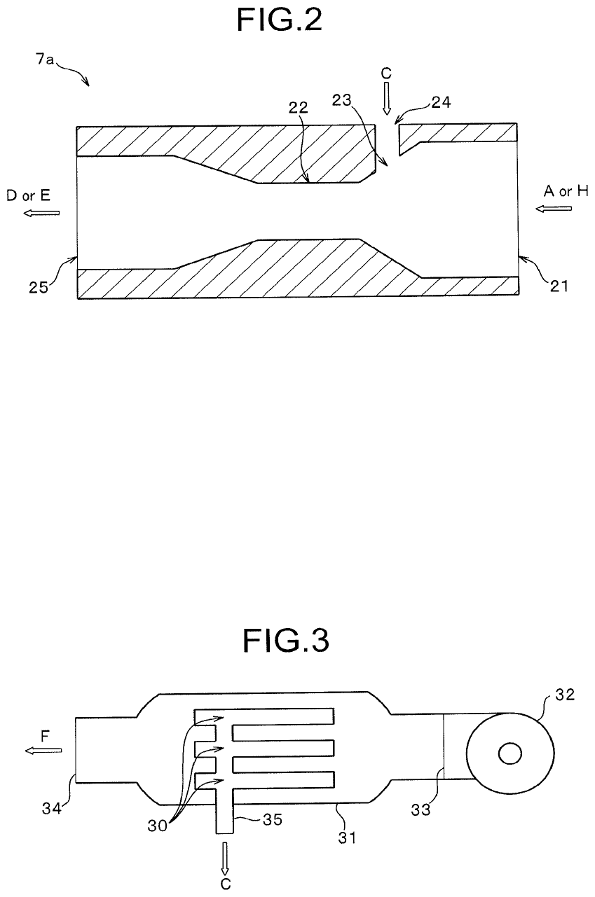

As the MNB generator in which the biological culture solution extracted from the culture tank is admixed with MNB, a (water flow type) MNB generator that can economically generate a large amount of MNB and is driven by using a water stream can be preferably used. The MNB generator will be schematically described later in FIG. 2. First, a case of using such a water flow type MNB generator will be described.

In the water flow type MNB generator, the biological culture solution extracted from the culture tank is supplied with pressure, and the diameter of a pipe conduit is reduced to increase a flow rate, etc., thereby generating turbulent flow. By supplying a gas such as air to the turbulent flow, the MNB is generated by water flow.

By suppressing the return ratio to a low level, the stress and damage visited on the microorganisms, etc., can be reduced by liquid circulation. Meanwhile, with the reduction in the return ratio, water flow as a driving source that generates the MNB weakens in the water flow type MNB generator, whereby the generation amount of the MNB is reduced, and the dissolved oxygen concentration is reduced. The amount of the MNB that can be contained in the liquid has a limit naturally. This also reduces the amount of the MNB that can be supplied to the biological culture solution extracted from the culture tank per hour as the return amount is reduced, to reduce the dissolved oxygen concentration. Thus, mere suppression of the return ratio to a low level has a limitation in efficiently and economically performing the biological reaction using the microorganisms. etc., but this problem can be solved by increasing the oxygen partial pressure of the gas phase in the culture tank.

In the invention, in order to efficiently and economically perform the biological reaction using the water flow type MNB generator, the stress and damage visited on the microorganisms, etc., by the liquid circulation can be reduced by suppressing the return ratio to a low level, and the reduction in the dissolved oxygen concentration associated therewith can be secured by increasing the oxygen partial pressure of the gas phase in the culture tank.

The upper limit of the return ratio is less than 48%, preferably 40% or less, more preferably 30% or less, and most preferably 20% or less. If the return ratio is excessively increased to 48% or more, the stress and damage visited on the microorganisms, etc., by the liquid circulation are increased. The lower limit of the return ratio is 1% or more, and preferably 10% or more. It is not preferable to excessively reduce the return ratio to less than 1% because the generation amount of the MNB is excessively reduced.

In the invention, even when an MNB generator other than the water flow type MNB generator is used, the generation amount of the MNB by the MNB generator can be reduced to reduce the stress and damage visited on the microorganisms, etc., and the reduction in the dissolved oxygen concentration associated therewith can be secured by increasing the oxygen partial pressure of the gas phase in the culture tank.

Furthermore, in the invention, as means for compensating for the reduction in the dissolved oxygen concentration associated with the suppression of the return ratio to a low level, means for setting the oxygen content ratio of the gas forming MNB to be higher than an oxygen content ratio in the air (about 21%), i.e., “at least 23% to less than 60%” is preferably used in combination with means for setting the oxygen partial pressure of the gas phase in the culture tank to be higher than the oxygen partial pressure of air (about 0.21 atm) at normal pressure, i.e., “at least 0.23 atm to less than 0.6 atm”, and/or means for setting the pressure of the gas phase in the culture tank to be higher than normal pressure (1 atm), i.e., “at least 1.1 atm to less than 3.0 atm”.

In order to obtain a gas forming MNB having an increased oxygen content ratio, normally, known oxygen enriching means such as a PSA method and a VSA method using an adsorbent, a water electrolysis method, a cryogenic separation method, a membrane separation method, or a chemical adsorption method is preferably used to increase the oxygen content ratio of the gas. An oxygen-enriched membrane is preferably used from the economical viewpoint.

The upper limit of the oxygen content ratio of oxygen-enriched MNB is less than 60%, preferably 55% or less, more preferably 50% or less, and most preferably 45% or less. When the oxygen concentration of the oxygen-enriched MNB is excessively increased to 60% or more, the stress and damage visited on the microorganisms, etc., by the oxidizing action of oxygen are increased. The lower limit of the oxygen concentration of the oxygen-enriched MNB is 23% or more, preferably 25% or more, more preferably 27% or more, and most preferably 30% or more. When the oxygen concentration of the oxygen-enriched MNB is excessively reduced to less than 23%, the dissolved oxygen concentration is reduced, which makes it difficult to enhance the activity of the microorganisms, etc.

### Second Feature: Use of Positive Displacement Pump

In the second feature of the invention, the use of 1) means for setting the oxygen partial pressure of the gas phase in the culture tank to be higher than the oxygen partial pressure of air (about 0.21 atm) at normal pressure, i.e., “at least 0.23 atm to less than 0.6 atm”, and/or 2) means for setting the pressure of the gas phase in the culture tank to be higher than normal pressure (1 atm), i.e., “at least 1.1 atm to less than 3.0 atm” enables to suppress the return ratio to a low level while maintaining the dissolved oxygen concentration without reducing the dissolved oxygen concentration, whereby a positive displacement pump such as a tube pump, a diaphragm pump, a screw pump, or a rotary pump that causes relatively less stress and damage applied to the microorganisms, etc., can be preferably used as a pump for circulating the biological culture solution to the outside of the culture tank.

The use of such a positive displacement pump makes it possible to further reduce the stress and damage visited on the microorganisms, etc.

### Specific Examples of Biological Reaction Device of the Invention and Biological Reaction in which Biological Reaction Device is Used

Next, the biological reaction device including the features of the invention and the biological reaction method in which the biological reaction device is used will be described in detail.

In the biological reaction device of the invention and the biological reaction in which the biological reaction device is used, the biological culture solution extracted from the culture tank using an extracting pump or a return pump, etc., is admixed with MNB, and the biological culture solution admixed with the MNB is returned to the culture tank. The following two methods can be employed as a method for admixing the biological culture solution extracted from the culture tank with oxygen-enriched MNB:

1) A method in which a biological culture solution extracted from a culture tank is separated into a filtrate and a biological culture solution excluding the filtrate by a filter, and the filtrate is admixed with oxygen-enriched MNB; and

2) A method in which a biological culture solution extracted from a culture tank is directly admixed with oxygen-enriched MNB without using a filter.

Since the method of the above 1) blows the oxygen-enriched MNB into the filtrate that does not substantially contain microorganisms etc., advantageously, stress and damage are not visited on the microorganisms etc., in the oxygen-enriched MNB blowing step. Meanwhile, the stress and damage may be visited on the microorganisms etc., in the filtration step. The amount of the filtrate separated in the filtration step is small (the amount of the filtrate is normally about 1/10 to 1/100 of the amount of the biological culture solution extracted from the culture tank). In order to supply a sufficient amount of MNB to the biological culture solution in the culture tank, it may be necessary to increase the amount of the biological culture solution extracted from the culture tank, and to increase the amount of the MNB blown, which may increase the operation cost of the device and increase also the stress and damage visited on the microorganisms, etc.

Since the method of the above 2) blows the MNB into the biological culture solution admixed with the microorganisms, etc., and extracted from the culture tank, the stress and damage may be visited on the microorganisms, etc., in the MNB blowing step. However, the stress and damage may be reduced in the filtration step as in the method 1). Even when it is necessary to increase the amount of the MNB blown in the method of the above 1), the biological culture solution extracted from the culture tank is directly admixed with the MNB by the method of the above 2), which eliminates the need for increasing the amount of the biological culture solution, and prevents the increase in the operation cost of the device and increase in the stress and damage visited on the microorganisms, etc.

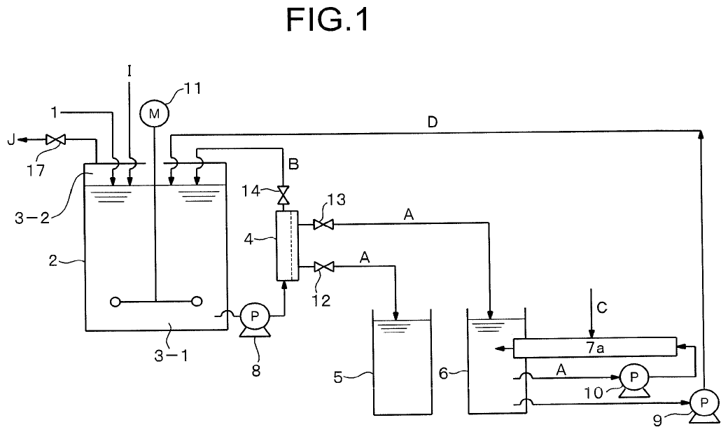

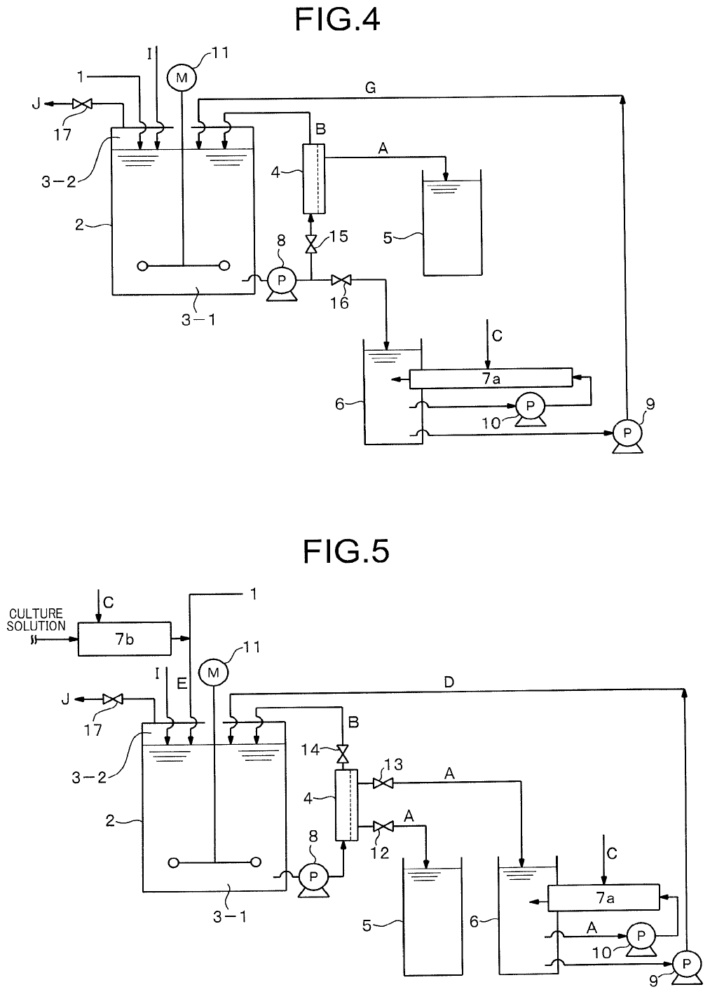

The biological reaction device employing the method of the above 1) will be described based on a first embodiment of the invention shown in FIG. 1, and the biological reaction device adopting the method of the above 2) will be described based on a second embodiment of the invention shown in FIG. 4.

### First Embodiment (FIG. 1)

First, a first embodiment of the invention will be described with reference to FIG. 1.

The first embodiment relates to a biological reaction device for causing microorganisms, etc., to produce a reaction product, wherein a biological culture solution is admixed with MNB according to the following steps:

a) supplying a culture solution 1 to a culture tank 2;

b) driving a culture tank pump 8 with a valve 12 closed and valves 13 and 14 opened, to extract a biological culture solution 3-1 containing a culture solution and microorganisms, etc., from the culture tank 2, and supplying the biological culture solution 3-1 to a filter 4;

c) returning a biological culture solution B (that is, a biological culture solution in which microorganisms, etc., are concentrated) separated by the filter 4 and excluding a filtrate to the culture tank 2;

d) accommodating a filtrate A separated by the filter 4 in an MNB generation tank 6, and causing an MNB generator 7a to admix the filtrate A with MNB;

g) driving a return pump 9 to return a filtrate D admixed with MNB to the culture tank 2;

h) advancing a biological reaction while the biological culture solution 3-1 in the culture tank 2 is stirred by a culture tank stirrer 11 in this way; and

i) driving the culture tank pump 8 with the valve 13 closed and the valves 12 and 14 opened when the biological reaction is sufficiently advanced, to recover a reaction product produced in the culture tank 2 together with the filtrate A, and to accommodate the reaction product and the filtrate A in a filtrate reservoir 5.

The filter 4 includes a filtration membrane and a container accommodating the filtration membrane. The filtration membrane may be either an organic membrane or an inorganic membrane. The filtration membrane to be employed may have any shape such as a flat membrane shape, a hollow fiber membrane shape, and a spiral shape. Among these, a hollow fiber membrane module is preferable, and the hollow fiber membrane module to be employed may be of an external pressure type or an internal pressure type.

A filtration system is preferably cross flow filtration using a hollow fiber membrane module. In this filtration system, a culture solution containing a reaction product and microorganisms, etc., is filtered while being supplied into a hollow fiber membrane, to take out a filtrate from the outside. Since the membrane fouling of the microorganisms, etc., deposited in the hollow fiber membrane is scraped off by shear power provided by the parallel flow of the culture solution, a stable filtration state can be maintained over a prolonged period.

When the cross flow filtration using the hollow fiber membrane module is performed, it is necessary to flow a liquid to be filtered into the hollow fiber membrane at a flow rate of a certain level or more in order to scrape off the membrane fouling. However, in the invention, the biological culture solution containing microorganisms, etc., to be filtered contains with oxygen-enriched MNB, which makes it possible to scrape off the membrane fouling even if the biological culture solution is flowed at a flow rate lower than usual, to allow stress and damage applied to the microorganisms, etc., to be largely reduced.

Specifically, general cross flow filtration is subjected to a steady operation at a circulating flow rate of about 1 to 2 m/s when the organic membrane is used, or about 1 to 3 m/s when a ceramic membrane is used. However, the biological culture solution is admixed with oxygen-enriched MNB, whereby the membrane fouling can be reduced, and the filtration resistance can be maintained at a low level. This can reduce a circulating flow rate required for obtaining the same flux (membrane filtered water volume per unit time/unit membrane area) to about 0.2 to 1.5 m/s. When the cross flow filtration is subjected to an operation at the same circulating flow rate, a flux can be increased by about 1.2 to 2.0 times.

As the filtration membrane, an organic polymer compound can be preferably used from the viewpoints of separation performance, water permeability, and fouling resistance. Examples thereof include a polyethylene-based resin, a polypropylene-based resin, a polyvinyl chloride-based resin, a polyvinylidene fluoride-based resin, a polysulfone-based resin, a polyether sulfone-based resin, a polyacrylonitrile-based resin, a cellulose-based resin, and a cellulose triacetate-based resin. A composite of resins containing these resins as a main component may be used. A polyvinyl chloride-based resin, a polyvinylidene fluoride-based resin, a polysulfone-based resin, a polyether sulfone-based resin, and a polyacrylonitrile-based resin are preferable, in which membrane formation using a solution is easy and which are excellent in physical durability and chemical resistance. A polyvinylidene fluoride-based resin or a resin containing the polyvinylidene fluoride-based resin as a main component is more preferably used since it is characterized by having both chemical strength (particularly, chemical resistance) and physical strength.

Here, as the polyvinylidene fluoride-based resin, a homopolymer of vinylidene fluoride is preferably used. Furthermore, as the polyvinylidene fluoride-based resin, a copolymer having vinylidene fluoride and a copolymerizable vinyl monomer may be used. Examples of the vinyl monomer copolymerizable with vinylidene fluoride include tetrafluoroethylene, hexafluoropropylene, and trichlorofluoroethylene.

The average micropore diameter of the filtration membrane can be appropriately determined according to purpose and situation to be used. The average micropore diameter is preferably rather smaller, and is normally 0.01 μm or more and 1 μm or less. When the average micropore diameter of the hollow fiber membrane is less than 0.01 μm, microorganisms, etc., and a membrane fouling component such as a component including saccharides and proteins and an aggregated body thereof block the micropores, and a stable operation cannot be performed. In view of balance with the water permeability, the average micropore diameter is preferably 0.02 μm or more, and more preferably 0.03 μm or more. When it exceeds 1 μm, the dirt component is insufficiently peeled from the micropores by shear power due to smoothness of and flow on a membrane surface and physical cleaning such as backwashing and air scrubbing. Therefore, a stable operation cannot be performed.

When the average micropore diameter of the hollow fiber membrane is closed to the size of a microorganism, etc., the microorganism, etc., may directly block the micropores. Furthermore, some microorganisms or cultured cells in a fermentation broth may be killed to produce fracturing matters of the cells. The average micropore diameter is preferably 0.4 μm or less, and more preferably 0.2 μm or less, to prevent the blocking of the micropores caused by the fracturing matters.

Here, the average micropore diameter of the filtration membrane can be calculated by measuring the diameters of a plurality of micropores observed under a scanning electron microscope at a magnification of 10,000 times or more and averaging the diameters. It is preferable that the average micropore diameter is calculated by randomly selecting 10 or more, and preferably 20 or more micropores, measuring the diameters of these micropores, and number-averaging the diameters. When the micropore is not circle, it is preferable that a circle having an area equivalent to the area of the micropore, that is, an equivalent circle, is determined with an image processing apparatus, etc., and the average micropore diameter is determined through a method using the diameter of the equivalent circle as the diameter of the micropore.

As shown in FIG. 1, in the first embodiment, the filtrate A that is a liquid to be admixed with MNB is extracted from the MNB generation tank 6 by driving a solution supply pump 10, and supplied to the MNB generator 7a. A gas C forming MNB is supplied to the MNB generator 7a.

As the MNB generator 7a used in the first embodiment, as schematically shown in FIG. 2, an MNB generator of a system (water stream system) that can economically generate a large amount of MNB and is driven by using a water stream is used. In the MNB generator 7a, the filtrate A is supplied from an inlet port part 21 of a nozzle in a pressurized state, and turbulent flow is generated in a throat part 22 while the diameter of a pipe conduit is reduced to increase a flow rate. In this state, the gas C forming MNB is supplied from a gas inlet port 24, and mixed with the filtrate A in a suction part 23 to form MNB according to water flow. A filtrate D containing MNB is discharged from an outlet port part 25, and supplied to the MNB generation tank 6.

By adjusting the flow rates of the filtrate A and the gas C forming MNB to be supplied to the MNB generator 7a, the amount and size of the MNB can be adjusted.

In the first embodiment, in order to reduce the stress and damage visited on the microorganisms, etc., the amount of the biological culture solution 3-1 extracted from the culture tank 2 is reduced to “at least 1% to less than 48% of the biological culture solution accommodated in the culture tank 2 per minute”, and the reduction in the dissolved oxygen concentration associated therewith can be compensated for by using 1) means for setting the oxygen partial pressure of the gas phase 3-2 in the culture tank 2 to be higher than the oxygen partial pressure of air (about 0.21 atm) at normal pressure, i.e., “at least 0.23 atm to less than 0.6 atm”, and/or 2) means for setting the pressure of the gas phase 3-2 in the culture tank 2 to be higher than normal pressure (1 atm), i.e., “at least 1.1 atm to less than 3.0 atm”. Furthermore, 3) means for setting the oxygen content ratio of the gas C forming MNB to be higher than an oxygen content ratio in air (about 21%), i.e., “at least 23% to less than 60%” may be used in combination with the means of the above 1) or 2).

The means of the above 1) to 3) in the first embodiment will be described in more detail.

As the means of the above 1), various known means can be used. Suitable techniques thereof include:

1a′) a technique of supplying air (oxygen content ratio: about 21%) having pressure higher than normal pressure and having a normal composition to the gas phase 3-2 in the culture tank 2 from an air supply path I, and causing a pressure adjusting valve 17 provided in an exhaust path J to increase the pressure of the gas phase 3-2 in the culture tank 2, thereby increasing the oxygen partial pressure of the gas phase 3-2 in the culture tank 2;

1b′) a technique of supplying air having normal pressure and having an increased oxygen content ratio to the gas phase 3-2 in the culture tank 2 from an air supply path I, and increasing the oxygen content ratio of the gas phase 3-2 in the culture tank 2 in a state where a pressure adjusting valve 17 provided in an exhaust path J is opened, to increase the oxygen partial pressure of the gas phase 3-2 in the culture tank 2; and

1c′) a technique of supplying air having pressure higher than normal pressure and an increased oxygen content ratio to the gas phase 3-2 in the culture tank 2 from an air supply path I, and causing a pressure adjusting valve 17 provided in an exhaust path J to increase the pressure and oxygen content ratio of the gas phase 3-2 in the culture tank 2, thereby increasing the oxygen partial pressure of the gas phase 3-2 in the culture tank 2.

As the means of the above 2), various known means can be used, and suitable examples thereof include a technique of supplying a gas having pressure higher than normal pressure to the gas phase 3-2 in the culture tank 2 as in the techniques of the above 1a′) and 1c′), to increase the pressure of the gas phase in the culture tank. In this technique, a gas having pressure higher than normal pressure can also be directly supplied to the gas phase 3-2 in the culture tank 2, or can also be supplied to the MNB generator 7a as the gas C forming MNB.

For the means of the above 3), a gas having an oxygen content ratio increased by using the known oxygen enriching means can be used as the gas C forming MNB. Specifically, oxygen-enriched air obtained by using an oxygen-enriched membrane as shown in FIG. 3 can be used as the gas C forming MNB.

In the oxygen enriching means using this oxygen-enriched membrane, basically, a container 31 including an oxygen-enriched membrane 30 includes a gas introduction part 33 and a derivation part 34 discharging a gas F having a low oxygen content ratio at both ends. A gas pressurized by an intake fan 32 is flowed to the oxygen-enriched membrane 30 from the gas introduction part 33, and the gas C having an increased oxygen content ratio is discharged from a derivation part 35. The gas F having a low oxygen content ratio is discharged from the derivation part 34.

In the first embodiment, a positive displacement pump such as a tube pump, a diaphragm pump, a screw pump, or a rotary pump that causes relatively less stress and damage applied to the microorganisms, etc., can be preferably used as the culture tank pump 8 and the return pump 9. This can also further reduce the stress and damage visited on the microorganisms, etc.

Furthermore, energy required for driving an MNB generator can be reduced by reducing the amount of the MNB contained in the culture solution.

### Second Embodiment (FIG. 4)

Next, a second embodiment of the invention will be described with reference to FIG. 4.

The second embodiment relates to a biological reaction device for causing microorganisms, etc., to produce a reaction product, wherein a biological culture solution is admixed with oxygen-enriched MNB according to the following steps:

a) supplying a culture solution 1 to a culture tank 2;

b) driving a culture tank pump 8 with a valve 15 closed and a valve 16 opened, to extract a biological culture solution 3-1 containing microorganisms, etc., from the culture tank 2, and supplying the biological culture solution 3-1 to an MNB generation tank 6;

c) accommodating the biological culture solution 3-1 in the MNB generation tank 6, and causing an MNB generator 7a to admix the biological culture solution 3-1 with MNB;

d) driving a return pump 9 to return a biological culture solution G admixed with MNB to the culture tank 2;

e) advancing a biological reaction while the biological culture solution 3-1 in the culture tank 2 is stirred by a culture tank stirrer 11 in this way; and

f) driving the culture tank pump 8 with the valve 16 closed and the valve 15 opened when the biological reaction is sufficiently advanced, to recover a reaction product produced in the culture tank 2 together with a filtrate A, and to accommodate the reaction product and the filtrate A in a filtrate reservoir 5.

As the method of the above 1) (first embodiment) and the method of the above 2) (second embodiment) as the method for admixing the biological culture solution extracted from the culture tank with MNB, it is preferable to employ a method in which the microorganisms, etc., are less stressed and damaged in total according to the kind of the microorganisms etc., and the condition of a biological reaction, etc.

In the second embodiment, as with the first embodiment, the amount of the biological culture solution 3-1 extracted from the culture tank 2 is reduced to reduce the stress and damage visited on the microorganisms, etc., and the reduction in the dissolved oxygen concentration associated therewith can be compensated for by using 1) means for setting the oxygen partial pressure of the gas phase 3-2 in the culture tank 2 to be higher than the oxygen partial pressure of air (about 0.21 atm) at normal pressure, i.e., “at least 0.23 atm to less than 0.6 atm”, and/or 2) means for setting the pressure of the gas phase 3-2 in the culture tank 2 to be higher than normal pressure (1 atm), i.e., “at least 1.1 atm to less than 3.0 atm”. Furthermore, 3) means for setting the oxygen content ratio of a gas C forming MNB to be higher than an oxygen content ratio in air (about 21%), i.e., “at least 23% to less than 60%” may be preferably used in combination with the means of the above 1) or 2).

Furthermore, the return ratio is suppressed to a low level, whereby the stress and damage visited on the microorganisms, etc., can be reduced, and energy required for circulating the biological culture solution can be reduced.

In the first embodiment and the second embodiment, as means for supplying MNB to the biological culture solution, means for admixing the biological culture solution extracted from the culture tank with oxygen-enriched MNB and returning the biological culture solution to the culture tank (hereinafter, referred to as “first means”) is used, but it is also possible to use other means in combination with this.

When the first means is used alone, it may take time to set the content of the oxygen-enriched MNB of the biological culture solution in the culture tank to an appropriate value. When it is necessary to shorten this time, means for admixing the culture solution supplied to the culture tank with oxygen-enriched MNB (hereinafter, referred to as “second means”), and means for admixing the biological culture solution in the culture tank with oxygen-enriched MNB (hereinafter referred to as “third means”), etc., are preferably used in combination. In particular, the second means is preferable as means to be used in combination with the first means because the stress and damage are not visited on the microorganisms, etc., by blowing the MNB.

### Third Embodiment (FIG. 5)

Next, a third embodiment of the invention will be described with reference to FIG. 5.

The third embodiment relates to a biological reaction device for causing microorganisms, etc., to produce a reaction product, wherein the second means is used in combination with the first embodiment using the first means.

In the third embodiment, a culture solution of microorganisms, etc., is admixed with oxygen-enriched MNB according to the following steps:

a) admixing a culture solution 1 supplied to a culture tank 2 with oxygen-enriched MNB by an MNB generator 7b;

b) advancing a biological reaction while a biological culture solution 3-1 in the culture tank 2 is stirred by a culture tank stirrer 11 in this way;

c) admixing a filtrate A obtained by filtering the biological culture solution 3-1 according to the procedures of b) to g) of the first embodiment with oxygen-enriched MNB when the dissolved oxygen concentration of the biological culture solution 3-1 is reduced, and returning the filtrate A to the culture tank 2 to adjust the dissolved oxygen concentration of the biological culture solution 3-1 to an appropriate value; and

d) driving a culture tank pump 8 with a valve 13 closed and valves 12 and 14 opened when the biological reaction is sufficiently advanced, to recover a reaction product produced in the culture tank 2 together with the filtrate A, and to accommodate the reaction product and the filtrate A in a filtrate reservoir 5.

In the third embodiment, as with the first embodiment and the second embodiment, the amount of the biological culture solution 3-1 extracted from the culture tank 2 can be reduced in order to reduce stress and damage visited on the microorganisms, etc., and the reduction in the dissolved oxygen concentration associated therewith can be compensated for by using 1) means for setting the oxygen partial pressure of a gas phase 3-2 in the culture tank 2 to be higher than the oxygen partial pressure of air (about 0.21 atm) at normal pressure, i.e., “at least 0.23 atm to less than 0.6 atm”, and/or 2) means for setting the pressure of the gas phase 3-2 in the culture tank 2 to be higher than normal pressure (1 atm), i.e., “at least 1.1 atm to less than 3.0 atm”. Furthermore, 3) means for setting the oxygen content ratio of a gas C forming MNB to be higher than an oxygen content ratio in air (about 21%), i.e., “at least 23% to less than 60%” may be preferably used in combination with the means of the above 1) or 2).

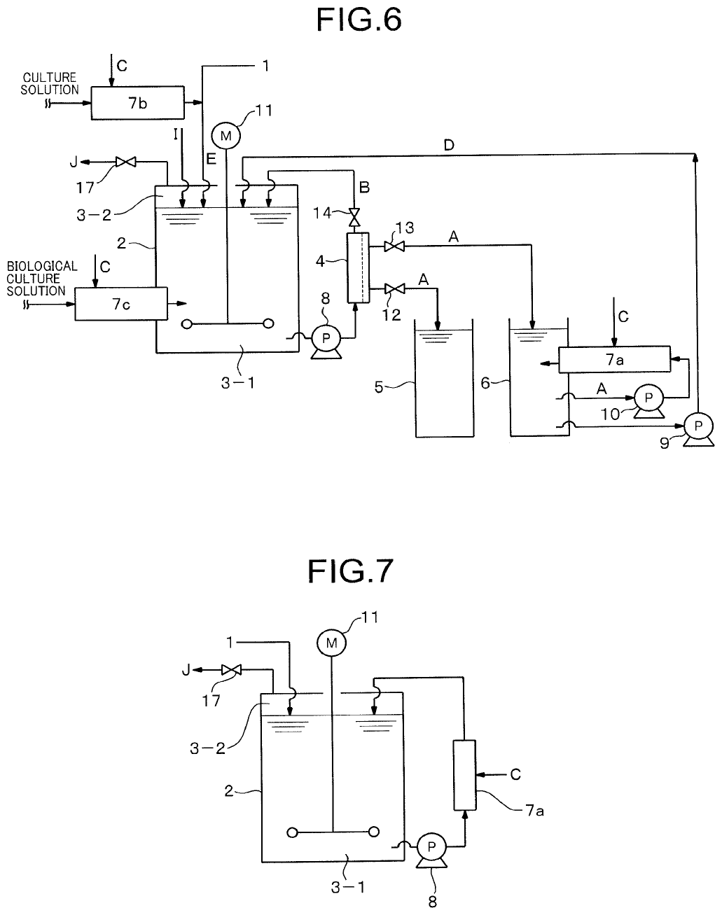

### Fourth Embodiment (FIG. 6)

Next, a fourth embodiment of the invention will be described with reference to FIG. 6.

The fourth embodiment relates to a biological reaction device for causing microorganisms, etc., to produce a reaction product, wherein the second means and the third means are used in combination with the first embodiment using the first means.

In the fourth embodiment, a culture solution of microorganisms, etc., is admixed with oxygen-enriched MNB according to the following steps:

a) admixing a culture solution 1 supplied to a culture tank 2 by an MNB generator 7b with oxygen-enriched MNB;

b) advancing a biological reaction while a biological culture solution 3-1 in the culture tank 2 is stirred by a culture tank stirrer 11 in this way;

c) admixing a filtrate A obtained by filtering the biological culture solution 3-1 according to the procedures of b) to g) of the first embodiment with oxygen-enriched MNB when the dissolved oxygen concentration of the biological culture solution 3-1 is reduced, and returning the filtrate A to the culture tank 2, or causing a MNB generator 7c to admix the biological culture solution 3-1 in the culture tank 2 with oxygen-enriched MNB, to adjust the dissolved oxygen concentration of the biological culture solution 3-1 to an appropriate value; and

d) driving a culture tank pump 8 with a valve 13 closed and valves 12 and 14 opened when the biological reaction is sufficiently advanced, to recover a reaction product produced in the culture tank 2 together with the filtrate A, and to accommodate the reaction product and the filtrate A in a filtrate reservoir 5.

In the fourth embodiment, as with the first embodiment, the second embodiment, and the third embodiment, the amount of the biological culture solution 3-1 extracted from the culture tank 2 can be reduced to reduce stress and damage visited on the microorganisms, etc., and the reduction in the dissolved oxygen concentration associated therewith can be compensated for by using 1) means for setting the oxygen partial pressure of the gas phase 3-2 in the culture tank 2 to be higher than the oxygen partial pressure of air (about 0.21 atm) at normal pressure, i.e., “at least 0.23 atm to less than 0.6 atm”, and/or 2) means for setting the pressure of the gas phase 3-2 in the culture tank 2 to be higher than normal pressure (1 atm), i.e., “at least 1.1 atm to less than 3.0 atm”. Furthermore, 3) means for setting the oxygen content ratio of a gas C forming MNB to be higher than an oxygen content ratio in air (about 21%), i.e., “at least 23% to less than 60%” may be preferably used in combination with the means of the above 1) or 2).

As the third embodiment and the fourth embodiment of the invention, the second means, and the second means and the third means in combination with the first embodiment of the invention (using the first means) have been described. Similarly, the second means, and the second means and the third means can be used in combination with the second embodiment of the invention (using the first means). The exhibition of the same function effect is readily apparent to a person skilled in the art.

### Fifth Embodiment (FIG. 7)

Next, a fifth embodiment of the invention will be described with reference to FIG. 7.

In the fifth embodiment, in order to reduce stress and damage visited on microorganisms, etc., 1) means for setting the oxygen partial pressure of a gas phase 3-2 in a culture tank 2 to be higher than the oxygen partial pressure of air (about 0.21 atm) at normal pressure, i.e., “at least 0.23 atm to less than 0.6 atm”, and/or 2) means for setting the pressure of the gas phase 3-2 in the culture tank 2 to be higher than normal pressure (1 atm), i.e., “at least 1.1 atm to less than 3.0 atm” are applied according to the following steps:

a) supplying a culture solution 1 to a culture tank 2;

b) driving a culture tank pump 8 to extract a biological culture solution 3-1 containing a culture solution and microorganisms, etc., from the culture tank 2 and supplying the biological culture solution 3-1 to an MNB generator 7a;

c) supplying air having normal pressure or pressure higher than the normal pressure, and having a normal composition or an increased oxygen content ratio as a gas C forming MNB to the MNB generator 7a to admix the biological culture solution 3-1 with MNB;

d) returning the biological culture solution 3-1 admixed with MNB is returned to the culture tank 2; and

e) advancing a biological reaction while the biological culture solution 3-1 in the culture tank 2 is stirred by a culture tank stirrer 11 in this way.

As the MNB generator 7a used in the fifth embodiment, as schematically shown in FIG. 2, an MNB generator of a system (water stream system) that can economically generate a large amount of MNB and is driven by using a water stream is used. In the MNB generator 7a, the filtrate A is supplied from an inlet port part 21 of a nozzle in a pressurized state, and turbulent flow is generated in a throat part 22 while the diameter of a pipe conduit is reduced to increase a flow rate. In this state, the gas C forming MNB is supplied from a gas inlet port 24, and mixed with the filtrate A in a suction part 23 to form MNB according to water flow. A filtrate D containing MNB is discharged from an outlet port part 25, and supplied to the MNB generation tank 6.

In the fifth embodiment, the amount of the biological culture solution 3-1 extracted from the culture tank 2 can be reduced to reduce the stress and damage visited on the microorganisms, etc., and the reduction in the dissolved oxygen concentration associated therewith can be compensated for by using 1) means for supplying air having an ordinary composition or air having an increased oxygen content ratio as a gas C forming MNB to the MNB generator 7a to set the oxygen partial pressure of the gas phase 3-2 in the culture tank 2 to be higher than the oxygen partial pressure of air (about 0.21 atm) at normal pressure, i.e., “at least 0.23 atm to less than 0.6 atm”, and/or 2) means for setting the pressure of the gas phase 3-2 in the culture tank 2 to be higher than normal pressure (1 atm), i.e., “at least 1.1 atm to less than 3.0 atm”. Furthermore, 3) means for setting the oxygen content ratio of the gas C forming MNB to be higher than an oxygen content ratio in air (about 21%), i.e., “at least 23% to less than 60%” may be used in combination with the means of the above 1) or 2).

Suitable examples of specific means of the above 1) include:

1a″) a technique of supplying air having pressure higher than normal pressure and having a normal composition (oxygen content ratio: about 21%) to the gas inlet port 24 of the MNB generator 7a, to increase the pressure of the gas phase 3-2 in the culture tank 2, thereby increasing the oxygen partial pressure of the gas phase 3-2 in the culture tank 2;

1b″) a technique of supplying air having normal pressure and having an increased oxygen content ratio to the gas inlet port 24 of the MNB generator 7a, to increase the oxygen content ratio of the gas phase 3-2 in the culture tank 2, thereby increasing the oxygen partial pressure of the gas phase 3-2 in the culture tank 2; and

1c″) a technique of supplying air having pressure higher than normal pressure and an increased oxygen content ratio to the gas inlet port 24 of the MNB generator 7a to increase the pressure and the oxygen content ratio of the gas phase 3-2 in the culture tank 2, thereby increasing the oxygen partial pressure of the gas phase 3-2 in the culture tank 2.

As specific means of the above 2), the techniques of the above 1a″) and 1c″) can be preferably used.

Examples of specific means of the above 3) include use of oxygen enriched air obtained by using the oxygen-enriched membrane as schematically shown in FIG. 3, as the gas C forming MNB.

As described above, in the biological reaction device of the invention and the biological reaction method in which the biological reaction device is used, the use of

1) means for setting the oxygen partial pressure of the gas phase in the culture tank to be higher than the oxygen partial pressure of air (about 0.21 atm) at normal pressure, i.e., “at least 0.23 atm to less than 0.6 atm”, and/or

2) means for setting the pressure of the gas phase in the culture tank to be higher than normal pressure (1 atm), i.e., “at least 1.1 atm to less than 3.0 atm” makes it possible to maintain the dissolved oxygen concentration without reducing the dissolved oxygen concentration even when the return amount is reduced to “at least 1% to less than 48% of the biological culture solution accommodated in the culture tank per minute” in order to reduce the stress and damage visited on the microorganisms, etc., whereby the activity of the microorganisms, etc., can be enhanced. Furthermore, preferably, 3) means for setting the oxygen content ratio of the gas forming MNB to be higher than the oxygen content ratio in air (about 21%), i.e., “at least 23% to less than 60%” is used in combination with the means of the above 1) or 2), whereby the above effect can be further exhibited.

A positive displacement pump such as a tube pump, a diaphragm pump, a screw pump, or a rotary pump that causes relatively less stress and damage applied to the microorganisms, etc., can be preferably used as a pump for circulating the biological culture solution to the outside of the culture tank such as a pump for extracting the biological culture solution from the culture tank, or a pump for returning the biological culture solution admixed with the oxygen-enriched MNB to the culture tank, which also can further reduce the stress and damage visited on the microorganisms, etc.

Furthermore, the return ratio is suppressed to a low level, whereby the stress and damage visited on the microorganisms, etc., can be reduced, and energy required for circulating the biological culture solution can be reduced.

Thus, the invention enables the biological reaction in which the microorganisms, etc., are used to be efficiently and economically performed, which is excellent.

The biological reaction device of the invention and the biological reaction method in which the biological reaction device is used reduce the stress and damage visited on the microorganisms, etc., in a biological reaction, and cause a biological reaction in which the microorganisms, etc., are used to be efficiently and economically performed under

1) a condition in which the return amount is set to “at least 1% to less than 48% of the biological culture solution accommodated in the culture tank per minute” and

2) a condition in which the oxygen partial pressure of the gas phase in the culture tank is set to “at least 0.23 atm to less than 0.6 atm” and/or the pressure of the gas phase in the culture tank is set to “at least 1.1 atm to less than 3.0 atm”.

However, the conditions of the above 1) and 2) may be maintained at the main stage of the biological reaction (the stage in which the production of the reaction product by the microorganisms etc., and the proliferation of the microorganisms etc. is performed in practice). Considering the economic performance and efficiency, etc., of the biological reaction at a stage that is trouble-free even if the speed of the biological reaction is slow such as an initial stage at which the number of the microorganisms is small and an end stage at which the number of the microorganisms is sufficiently increased, it is also possible to eliminate the conditions of the above 1) and 2).

The above-mentioned function effects of the invention will be described below using Reference Examples, Reference Comparative Examples, and theoretical formulae, etc., but the invention is not limited by these descriptions.

### Reference Examples 1 and 2/Reference Comparative Examples 1 to 5

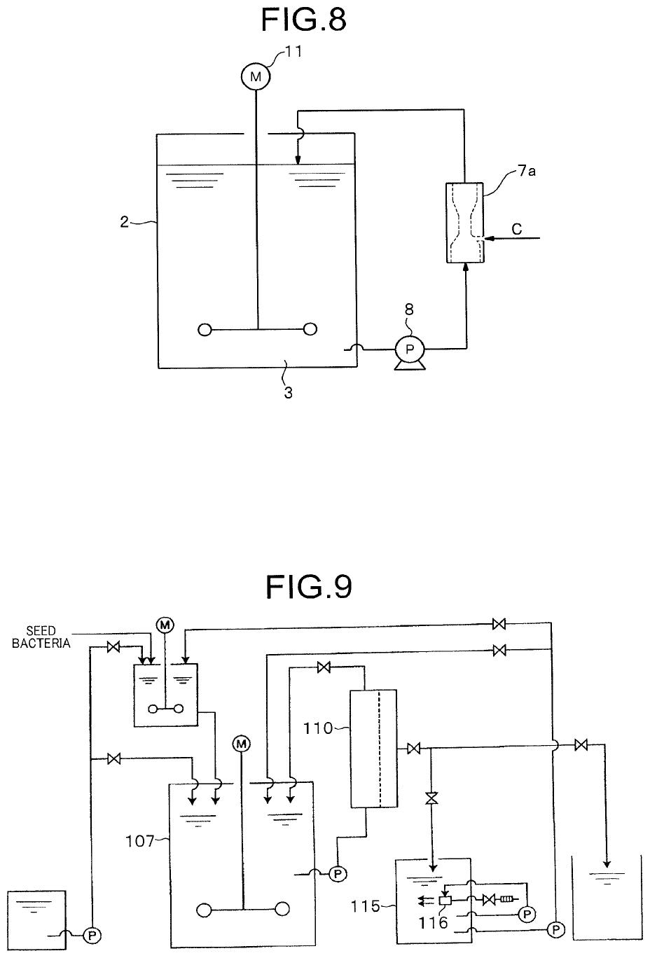

In the following Reference Examples 1 and 2 and Reference Comparative Examples 1 to 5, microorganisms were cultured using a device as schematically shown in FIG. 8.

As a culture tank 2, a microorganism culture device (a microorganism culture device BMZ-P manufactured by ABLE Corporation, internal volume: 1000 ml) was used, in which aerobic microorganisms [type culture of coryneform bacteria (Corynebacterium glutamicum)], and a biological culture solution 3 containing a culture solution [synthetic medium containing ammonium sulfate as a main component, glucose concentration: 4%] were accommodated so that the volume was set to 500 mL. The initial fungicidal concentration of the biological culture solution 3 had turbidity (value of OD 610):1.

While aerobic microorganisms are cultured with a culture temperature set to 33° C., culture pressure set to 1 atm, and the number of rotations of a culture tank stirrer 11 set to 600 rpm, a culture tank pump 8 is driven to take out the biological culture solution 3 in a fixed return amount from a culture tank 2. The biological culture solution 3 is supplied to a water flow type MNB generator 7a [water flow type MNB generator, manufactured by OK ENGINEERING:KK, model number: OKE-MB 200 ml] as schematically shown in FIG. 2, to admix the biological culture solution 3 with MNB, and the biological culture solution 3 was then returned to the culture tank 2. Air that is a gas C forming MNB having a constant oxygen content ratio was supplied to the MNB generator 7a at a flow rate of 250 mL/min.

Under such a condition, the aerobic microorganisms were cultured for 8 hours in a state where the return amount and the oxygen content ratio of the gas C forming MNB were changed, and the turbidity of the fungicidal concentration and the dissolved oxygen concentration of the biological culture solution 3 in the culture tank 2 after the lapse of 8 hours were measured to obtain Reference Examples 1 and 2 and Reference Comparative Examples 1 to 5. In Reference Examples 1 and 2 and Reference Comparative Examples 1 to 5, return amounts (mL/min), return ratios (vol %), MNB oxygen content ratios (mol %), fungicidal concentrations (turbidity: value of OD 660), and dissolved oxygen concentrations (mg/L) are set in order and shown in Table 1. The “return ratio” refers to a ratio of a return amount (mL/min) per minute to the amount (500 mL) of the biological culture solution 3 accommodated in the culture tank 2.

TABLE 1

| | | Reference | Reference | | | Reference | Reference | Reference |
|---|---|---|---|---|---|---|---|---|
| | | Comparative | Comparative | Reference | Reference | Comparative | Comparative | Comparative |
| | | Example 1 | Example 2 | Example 1 | Example 2 | Example 3 | Example 4 | Example 5 |
| A: Return | mL/min | 240 | 80 | 80 | 80 | 80 | 80 | 80 |
| amount | | | | | | | | |
| B: Return | vol % | 48 | 16 | 16 | 16 | 16 | 16 | 16 |
| ratio (A/500) | | | | | | | | |
| C: Oxygen | mol % | 21 | 21 | 30 | 40 | 60 | 80 | 100 |
| content ratio | | | | | | | | |
| of MNB | | | | | | | | |
| D: Fungicidal | OD 610 | 21 | 25 | 28 | 34 | 20 | 5 | 4 |
| concentration | | | | | | | | |
| E: Dissolved | mg/L | 6.9 | 1.2 | 2.6 | 5.8 | 23 | 31 | 38 |
| oxygen | | | | | | | | |
| concentration | | | | | | | | |

Reference Examples 1 and 2 and Reference Comparative Examples 1 to 5 will be described below.

First, in Reference Comparative Examples 1 and 2, air was supplied to the water flow type MNB generator 7a, to set the oxygen content ratio of the MNB to 21%. The return ratio was 48% in Reference Comparative Example 1, and 16% in Reference Comparative Example 2. Thus, since stress and damage visited on the aerobic microorganisms by liquid circulation can be reduced if the return ratio is suppressed to a low level, the fungicidal concentration can be increased to 25 (OD 610) from 21 (OD 610). Meanwhile, in a generally used water flow MNB generator such as the MNB generator 7a, the generation amount of the MNB itself is reduced if the return ratio is suppressed to a low level, whereby the dissolved oxygen concentration is reduced to 1.2 (mg/L) from 6.9 (mg/L).

Then, in Reference Examples 1 and 2 and Reference Comparative Examples 3 to 5, in a state where the return ratio was maintained at 16% as in Reference Comparative Example 2, and the stress and damage visited on the aerobic microorganisms by liquid circulation were reduced, the dissolved oxygen concentration was increased by increasing the oxygen content ratio of the MNB to 30%, 40%, 60%, 80%, and 100%.

The relationship between the oxygen content ratio (mol %) of the MNB and the fungicidal concentration (OD 610) in Reference Examples 1 and 2 and Reference Comparative Examples 3 to 5 is set in order and shown in Table 2. In Table 2, a vertical axis represents the numerical values of the oxygen content ratio (mol %) of the MNB and fungicidal concentration (OD 610), and represents the oxygen content ratios (mol %) of the MNB and fungicidal concentrations (OD 610) of Reference Comparative Example 2, Reference Examples 1 and 2, and Reference Comparative Examples 3 to 5 as broken lines, respectively.

As can be seen from Table 2, as the oxygen content ratio of the MNB is increased from 21% (Reference Comparative Example 2) to 30% (Reference Example 1) to 40% (Reference Example 2), the fungicidal concentration tends to be increased. The fungicidal concentration shifts to reduction tendency with the oxygen content ratio of the MNB of around 40% as the peak. When the oxygen content ratio is 60% (Reference Comparative Example 3), the fungicidal concentration is lower than 21% (Reference Comparative Example 2). This is considered to be because the stress and damage are visited on the aerobic microorganisms by the oxidation action of oxygen when the oxygen content ratio of the MNB is too high.

From these Reference Examples 1 and 2 and Reference Comparative Examples 1 to 5, in order to efficiently and economically perform the biological reaction using the microorganisms, etc., it can be seen that

1) by suppressing the return ratio to a low level to reduce the stress and damage visited on the microorganisms, etc., by liquid circulation, and

2) by increasing the oxygen content ratio of the MNB to increase the dissolved oxygen concentration while avoiding the stress and damage visited on the microorganisms, etc., by oxygen, without causing the reduction in the dissolved oxygen concentration associated with the suppression of the return ratio to a low level,

the fungicidal concentration in the biological reaction using the microorganisms, etc., can be increased, and the biological reaction using the microorganisms, etc., can be efficiently and economically performed.

Next, a method for culturing microorganisms, etc., using oxygen-enriched MNB while satisfying the above items 1) and 2) will be specifically described. The culture conditions of the microorganisms, etc., depend on the kind of the microorganisms, etc., the scale of the culture device, and the structure of the culture device, etc. A description will be given by exemplifying a case where microorganisms, etc., are cultured by using a culture device for microorganisms, etc., used in Reference Examples 1 and 2 and Reference Comparative Examples 1 to 5, as shown in FIG. 8.

### 1. Regarding Above 1)

In the culture device for the microorganisms, etc., shown in FIG. 8, the biological culture solution 3 is extracted from the culture tank 2 by the culture tank pump 8, and supplied to the water flow type MNB generator 7a. The biological culture solution 3 is admixed with oxygen-enriched MNB by the MNB generator 7a, and is returned to the culture tank 2. The stress and damage are greatly visited on the microorganisms etc., when passing through the MNB generator 7a, whereby the “stress and damage visited on the microorganisms etc.” can be evaluated with “the pressure of the biological culture solution 3 in the inlet port of the MNB generator 7a” (hereafter, referred to as “inlet port pressure”) as an index.

Table 3 shows a graph obtained by plotting a return ratio (% by volume) as a lateral axis and inlet port pressure (MPa) as a vertical axis. The return ratio and the inlet port pressure are measured while the driving force of the culture tank pump 8 is changed in the culture device for the microorganisms, etc., shown in FIG. 8.

Since resistance to the stress and damage is different depending on the microorganisms, etc., to be cultured, appropriate inlet port pressure can be set from the beginning of the culture if the upper limit of the inlet port pressure appropriate for the kind of the microorganisms, etc., is examined in advance and a database is created. When such a database is not created, it is possible to set inlet port pressure that is initially considered to be appropriate, determine the amount of the stress and damage from the culture conditions, etc., of the microorganisms, etc., and adjust the inlet port pressure.

For example, the inlet port pressure was initially set to 0.075 MPa, but if the stress and damage visited on the microorganisms, etc., are large, and the culture does not proceed smoothly, the driving force of the culture tank pump 8 is reduced to perform adjustment so that the inlet port pressure is reset to 0.04 MPa. This adjustment can reduce the stress and damage visited on the microorganisms, etc., but the return ratio is reduced from 30% to 20%, whereby the dissolved oxygen concentration is reduced.

### 2. Regarding Above 2)

As described in the above 1), if the inlet port pressure is reduced to reduce the stress and damage visited on the microorganisms, etc., the return ratio is reduced and the dissolved oxygen concentration is reduced. In order to compensate for the reduction, it is necessary to reset the oxygen content ratio of the MNB to a high level.

The appropriate oxygen content ratio of the MNB can be set as follows.

First, regarding the dissolved oxygen concentration, the following general formula (1) is known.

OTR=KLA×(Cs−C) (1)

In this formula (1),

OTR is an oxygen transfer rate (mg/L·h);

KLA is a mass transfer capacity coefficient (/h);

Cs is saturated solubility of oxygen in water (mg/L); and

C: solubility of oxygen in water (mg/L).

Even when the inlet port pressure and the return ratio are reduced, in order to maintain the dissolved oxygen concentration at a constant value, it is necessary to maintain the OTR at a constant value.

The value of the OTR is determined by variables “KLA” and “Cs” and a constant “C”. Among these, “Cs” can be represented as the function of the oxygen content ratio as in the following general formula (2).

Cs=X×P÷H×MO₂ (2)

In this formula (2),

X is an oxygen content ratio in air (mole fraction);

P is operating pressure of a culture tank (atm);

H is a Henry's constant (atm·m³/mol); and

MO₂ is a molecular weight of oxygen (g/mol).

“KLA” can be represented as the function of the return ratio by determining the relationship with the return ratio in the culture device to be used. For example, Table 4 shows a graph obtained by plotting a return ratio (% by volume) as a lateral axis (x) and KLA (/h) as a vertical axis (y). In the culture device for the microorganisms, etc., shown in FIG. 8, the return ratio and the KLA are measured while the driving force of the culture tank pump 8 is changed. From this, it is possible to obtain an approximate formula such as formula (3).

y=aln(x)−b (3)

In this formula,

y is KLA (/h);

x is a return ratio (% by volume); and

a and b are constants.

### 3. Regarding Control by Above 1) and 2)

According to the above formulae (1) to (3), for example, the inlet port pressure was initially set to 0.075 MPa. The stress and damage visited on the microorganisms, etc., are large, and the culture does not proceed smoothly. When the driving force of the culture tank pump 8 is reduced and the inlet port pressure is adjusted to 0.04 MPa, the return ratio is reduced from 30% to 20%, and the dissolved oxygen concentration is reduced. In order to compensate for the reduction to constantly maintain the dissolved oxygen concentration, the increase level of the oxygen content ratio of the MNB to be required can be determined. The setting ratio when the oxygen content ratio is controlled is preferably 70% to 130%, more preferably 80% to 120%, still more preferably 90% to 110%, and most preferably 95% to 105%. The “arrangement ratio” refers to a ratio of a control setting value to a target value of the oxygen content ratio obtained by the formulae (1) to (3).

In the culture device for the microorganisms, etc., as shown in FIG. 8 based on the formulae (1) to (3), by controlling the device for driving the culture tank pump 8 and the device for supplying the oxygen-enriched air to the MNB generator 7a, the dissolved oxygen concentration can be automatically maintained at a constant value even when the inlet port pressure and the return ratio are reduced, whereby the biological reaction using the microorganisms, etc., can be efficiently and economically performed.

### Description According to Theoretical Formulae Based on Reference Examples 1 and 2 and Reference Comparative Examples 1 to 5

As shown in Tables 5 and 6 below, from the above Reference Examples 1 and 2 and Reference Comparative Examples 1 to 5, when the return amount was set to a low level of 80 mL/min, and the return ratio was set to a low level of 16%, and the stress and damage visited on the microorganisms, etc., by liquid circulation are reduced, the fungicidal concentration in the biological reaction using the microorganisms, etc., can be maintained at a high level by increasing the dissolved oxygen concentration, and the biological reaction using the microorganisms, etc., can be efficiently and economically performed.

TABLE 5

| | | | | | | | |
|---|---|---|---|---|---|---|---|
| E: Dissolved | mg/L | 1.2 | 2.6 | 5.8 | 23 | 31 | 38 |
| oxygen | | | | | | | |
| concentration | | | | | | | |
| D: Fungicidal | OD 610 | 25 | 28 | 34 | 20 | 5 | 4 |
| concentration | | | | | | | |

In Reference Examples 1 to 2 and Reference Comparative Examples 1 to 5, the dissolved oxygen concentration is increased by means for setting the oxygen content ratio of the MNB to a high level, but the dissolved oxygen concentration can be similarly increased also by means for setting the oxygen partial pressure of the gas phase in the culture tank to be higher than the oxygen partial pressure of air (about 0.21 atm).

Theoretically, the change in the dissolved oxygen concentration of the culture solution of bacteria cells (dCa/dt) can be represented by the following formula (4).

dCa/dt=KLa(C*−Ca)−QO₂×Y (4)

In the above formula (4),

Ca represents a dissolved oxygen concentration of a culture solution (mg/L);

t represents an elapsed time (s);

KLa represents a mass transfer capacity coefficient (/s);

C* represents a saturated oxygen concentration of a culture solution (mg/L);

QO₂ represents a respiration rate per unit bacteria cell weight (mg/L⁻kg⁻s); and

Y represents a weight of bacteria cells in a culture solution (kg).

In the formula (4), a first term “KLa(C*−Ca)” on a right side represents the supply of dissolved oxygen to the culture solution, and a second term “QO₂×Y” on the right side represents the consumption of dissolved oxygen by bacteria cells. In order to increase the amount of dissolved oxygen, it is necessary to increase the value of “KLa(C*−Ca)”.

“KLa(C*−Ca)” is represented by the following formula (5):

KLa(C*−Ca)=KL×a×(p÷H×MO₂−C) (5)

wherein:

KL represents a mass transfer coefficient (m/s);

a represents a gas-liquid interface area in a culture solution (m²/m³);

p represents oxygen partial pressure (atm);

H represents a Henry's constant (atm·m³/mol); and

MO₂ represents a molecular weight of oxygen (g/mol).

It is found that, in order to increase the amount of the dissolved oxygen, the following techniques are effective:

1) a technique of increasing a (the gas-liquid interface area in the culture solution) using MNB;

2) a technique of increasing the oxygen content ratio of the gas forming MNB to increase p (oxygen partial pressure); and

3) a technique of increasing the oxygen partial pressure of the gas phase in the culture tank to increase p (oxygen partial pressure).

Both the previous invention and the invention use the technique of the above 1). In the previous invention, together with the technique of the above 1), the means for setting the oxygen content ratio of the gas forming MNB to be higher than an oxygen content ratio in air (about 21%) [the technique of the above 2)] is used. However, in the invention, together with the technique of the above 1), means for setting the oxygen partial pressure of the gas phase in the culture tank to be higher than the oxygen partial pressure of air (about 0.21 atm) [the technique of the above 3)] is used. In the invention, together with the technique of the above 1), the means for setting the oxygen partial pressure of the gas phase in the culture tank to be higher than the oxygen partial pressure of air (about 0.21 atm) [the technique of the above 3] and the means for setting the oxygen content ratio of the gas forming MNB to be higher than the oxygen content ratio in air (about 21%) [the technique of the above 2)] can also be used.

As shown in Tables 5 and 6 above, from the above Reference Examples 1 and 2 and Reference Comparative Examples 1 to 5, when the return amount is set to a low level of 80 mL/min, and the return ratio is set to a low level of 16%, and the stress and damage visited on the microorganisms, etc., by liquid circulation are reduced, the fungicidal concentration can be increased if the dissolved oxygen concentration amount is compensated for to about 5 mg/L to 15 mg/L. Therefore, it is apparent that, by the means for setting the oxygen partial pressure of the gas phase in the culture tank of the invention to be higher than the oxygen partial pressure of air (about 0.21 atm), the dissolved oxygen concentration amount can be compensated for to increase the fungicidal concentration.

### REFERENCE SIGNS LIST

1 culture solution
2 culture tank
3 biological culture solution (containing culture solution, microorganisms, etc.)
3-1 biological culture solution (containing culture solution, microorganisms, etc.)
3-2 gas phase in culture tank
4 filter
5 filtrate reservoir
6 MNB generation tank
7a to 7c MNB generator
8 culture tank pump
9 return pump
10 solution supply pump
11 culture tank stirrer
12 to 16 valve
17 pressure adjusting valve
21 inlet port part
22 throat part
23 suction part
24 gas inlet port
25 outlet port part
30 oxygen-enriched membrane
31 container
32 intake fan
33 gas introduction part
34 derivation part (exhausting gas having low oxygen content ratio)
35 derivation part (exhausting gas having increased oxygen content ratio)
A filtrate
B biological culture solution excluding filtrate
C gas forming MNB (gas or air having increased oxygen content ratio)
D filtrate admixed with MNB (filtrate+MNB)
E culture solution admixed with MNB (culture solution+MNB)
F gas having low oxygen content ratio
G biological culture solution admixed with MNB (biological culture solution+MNB)
H biological culture solution
I air supply path (to gas phase in culture tank)
J exhaust path (from gas phase in culture tank)
107 culture tank as biological reaction tank
110 bacteria cell filter
115 MNB generation tank
116 MNB generator

1. A biological reaction device, comprising:
a culture tank that accommodates a culture solution and a biological culture solution comprising aerobic or facultative anaerobic microorganisms;
a micro-nano-bubble generator for admixing a biological culture solution extracted from the culture tank with micro-nano-bubbles; and
a pipe conduit for returning a biological culture solution admixed with the micro-nano-bubbles to the culture tank,
wherein an amount per minute of the biological culture solution extracted from the culture tank and then returned to the culture tank after being admixed with the micro-nano-bubbles is at least 1% to less than 48% of the biological culture solution accommodated in the culture tank; and
an oxygen partial pressure of a gas phase in an upper part of the biological culture solution in the culture tank is at least 0.23 atm to less than 0.6 atm and/or a pressure of the gas phase in the upper part of the biological culture solution in the culture tank is at least 1.1 atm to less than 3.0 atm.

2. The biological reaction device of claim 1, wherein the micro-nano-bubbles are formed of a gas having an oxygen content ratio of at least 23% to less than 60%.

3. The biological reaction device of claim 1, wherein the micro-nano-bubble generator admixes the biological culture solution extracted from the culture tank using an extracting pump or a return pump with the micro-nano-bubbles, and returns the biological culture solution to the culture tank.

4. The biological reaction device of claim 1, wherein:
a filter is disposed between the culture tank and the micro-nano-bubble generator, to separate the biological culture solution extracted from the culture tank into a filtrate and a biological culture solution excluding the filtrate;
the filtrate is admixed with micro-nano-bubbles by the micro-nano-bubble generator; and
a pipe conduit is provided to return the biological culture solution excluding the filtrate and the filtrate admixed with the micro-nano-bubbles to the culture tank.

5. The biological reaction device of claim 1, wherein the biological culture solution extracted from the culture tank is directly admixed with the micro-nano-bubbles without interposing a filter between the culture tank and the micro-nano-bubble generator.

6. The biological reaction device of claim 1, wherein the micro-nano-bubble generator is of a system driven by using water flow.

7. The biological reaction device of claim 1, wherein a positive displacement pump is used as a pump for extracting the biological culture solution from the culture tank and/or as a pump for returning the biological culture solution admixed with the micro-nano-bubbles to the culture tank.

8. The biological reaction device of claim 7, wherein the positive displacement pump is a tube pump.

9. The biological reaction device of claim 2, wherein the gas having the oxygen content ratio is obtained by causing air to pass through an oxygen-enriched membrane.

10. The biological reaction device of claim 2, wherein the gas having the oxygen content ratio is obtained by mixing oxygen produced by any of a PSA method, a VSA method, a cryogenic separation method, and a chemical adsorption method.

11. The biological reaction device of claim 1, further comprising a micro-nano-bubble generator for admixing the culture solution supplied to the culture tank with micro-nano-bubbles formed from air having an increased oxygen content ratio.

12. The biological reaction device of claim 1, further comprising a micro-nano-bubble generator for admixing the biological culture solution in the culture tank with micro-nano-bubbles formed from air having an increased oxygen content ratio.

13. A biological reaction method for providing a reaction product of aerobic or facultative anaerobic microorganisms, or proliferating the aerobic or facultative anaerobic microorganisms, the method comprising operating the biological reaction device of claim 1.

14. The biological reaction device of claim 7, wherein the positive displacement pump is a tube pump, a diaphragm pump, a screw pump, or a rotary pump.

15. The biological reaction device of claim 10, wherein the mixing is carried out with a line mixer.
